(19) United States
(12) Patent Application Publication
(10) Pub. No.: US 2003/0013726 A1
(43) Pub. Date: Jan. 16, 2003

(54) Transdermal therapeutic system for the administration of zaleplon

(76) Inventor: Thorsten Selzer, Neuwied (DE)

Correspondence Address: BIRCH STEWART KOLASCH & BIRCH, PO BOX 747, FALLS CHURCH, VA, 22040-0747, US

(21) Appl. No.: 10/182,669

(22) PCT Filed: Jan. 18, 2001
(86) PCT No.: PCT/EP01/00532

(30) Foreign Application Priority Data
Feb. 1, 2000 (DE) 10004790.4

(51) Int. Cl. A61K 31/519

(52) U.S. Cl. 514/259.3

(57) ABSTRACT

A zaleplon-containing pharmaceutical preparation is a transdermal therapeutic system in plaster form, having a backing layer, having a contact adhesive active ingredient reservoir connected thereto, and having a protective layer which can be detached before application, where the active ingredient reservoir comprises zaleplon.

The invention relates to a pharmaceutical preparation with the active ingredient zaleplon in the form of a transdermal therapeutic system. The invention further encompasses the use of a zaleplon-containing transdermal therapeutic system for the treatment of prophylaxis of disease states or health disturbances, and to a process for producing such transdermal therapeutic systems.

Transdermal therapeutic systems (TTS) are dosage forms which are applied to the skin and are designed to make a drug substance systemically available after transdermal absorption. TTS may increase the therapeutic value of administration of a drug substance by ensuring a constant delivery of the active ingredient to the blood compartment over a prolonged period. Problems such as gastrointestinal intolerance, low enteral absorption, first-pass metabolism in the liver, and increased frequency of administration associated with low half-lives can thus be avoided.

Prior art TTS consist of a backing layer which is impermeable to drug substance, of a reservoir layer containing drug substance, and of a contact adhesive layer for attachment to the skin, it being possible for the latter to be identical to the reservoir layer containing drug substance, and of a protective layer which is to be removed before application and is impermeable to drug substance. The reservoir layer containing drug substance consists of drug substance (active ingredient) and excipients such as plasticizers, tackifiers, solubilizers, stabilizers, fillers, carriers and permeation promoters. The pharmaceutically acceptable substances suitable for this purpose are known to the skilled worker.

The active ingredient zaleplon, a pyrazolopyrimidine, is a novel hypnotic from the class of nonbenzodiazepines. The effects of zaleplon are explained by an agonistic effect on the benzodiazepine omega 1 (type 1) receptor subtype of the GABA(A) receptor complex (Scharf M., A new option for insomnia., Health News, Oct. 1, 1999, 5(12):4; Annseau M., Pharma-clinics, Drug of the month. Zaleplon. Rev. Med Liege 1999, August; 54(8):705-6).

Sleep is a continually repeated recuperative process of the body. It proceeds in various phases:

a) deep sleep with slowed electrical waves in the brain; this is also referred to as passive recuperative sleep. Regeneration and building-up processes take place in many organs during this phase.

b) dreaming sleep with flat electrical waves in the brain and rapid eye movements; this serves in particular for further processing of information taken in during the day.

In normal sleep the stage of falling asleep is followed by alternation four to five times of the two phases of sleep. Both types of sleep are absolutely necessary for physical and mental recuperation. Sleep disorders are present if this recuperative process is considerably impaired whether by a gradual change in the duration of sleep or in the progress of the phases of sleep.

About 10% of the population suffer from chronic and about 50% from occasional sleep disorders.

Zaleplon was investigated in a clinical study on more than 3 700 patients in the USA, Canada and Europe, including elderly people between 65 and 85 years of age. It proved to be very effective for patients with disorders of initiating sleep. Compared with placebo, in clinical studies it significantly reduced the sleep latency estimated both by polysomnography and subjectively. Sleep stages 3 and 4 (delta sleep and deep sleep) are not impaired by zaleplon. The effects on REM sleep, the total sleeping time and the sleep efficiency were variable in the individual studies and are possibly dose-dependent (12th Annual Meeting of the Assoc. Prof. Sleep Soc. Jul. 15, 1998, New Orleans).

However, zaleplon also has certain disadvantages which derive from the pharmacokinetics: zaleplon is rapidly absorbed after oral administration, but the absolute availability is only 30%. The plasma half-life is only one hour. The result of this may be that although oral administration of zaleplon shortens the sleep latency and brings about sleep onset, maintenance of sleep is not always ensured so that the patients wake up at times and may need to take another dose. It should be noted that sleep-promoting agents should always be administered in the lowest possible dose. It is therefore possible to only a very limited extent to increase the oral dose in order in particular to promote maintenance of sleep.

The object on which the present invention is based was to provide a zaleplon-containing drug preparation which avoids as far as possible the disadvantages which occur with oral administration of the active ingredient zaleplon and which have been described above. At the same time, it is intended that the drug preparation be suitable for the treatment of disorders of initiating and maintaining sleep.

This object is achieved according to the invention by a drug preparation as claimed in claim 1, according to which the zaleplon-containing drug preparation is a transdermal therapeutic system (TTS) in plaster form, where the active ingredient zaleplon is present in the active ingredient reservoir of the TTS. The TTS of the invention can be used in order to treat, via transdermal administration of zaleplon, disorders of initiating and/or maintaining sleep.

A prerequisite for the transdermal administration of zaleplon for the treatment of sleep disorders is that a high flux of active ingredient in vivo is achieved, which is the case with the transdermal preparations of the invention. On use of the zaleplon-containing TTS of the invention through application to the skin, the result is, unlike on oral administration, a substantially constant delivery of the active ingredient during the period of application. In this way there is compensation for the rapid elimination of the active ingredient zaleplon through continuous replenishment from the active ingredient reservoir of the TTS. In addition, the problem of oral availability is avoided in this way.

The transdermal administration of the sleep-promoting agent may be advantageous in particular for those patients who already need to take a relatively large number of various medicines orally because of other existing disorders. In addition, transdermal administration is also more reliable and controllable for the patient, e.g. if memory disturbances occur, which may in some circumstances lead to inadvertent repeated intake at short intervals of an agent which is to be administered orally, which might lead to an overdosage.

Administration of the pharmaceutical preparations of the invention can take place in such a way that appropriate TTS in the form plasters are stuck onto the skin about 2 h before going to bed and are removed again after getting up in the morning. Because of the rapid elimination and short half-life, the effect declines rapidly after removal of the plaster, so that no hangover effects result as occur with many other sleep-maintaining agents.

It is possible in principle for the TTS of the invention to be employed both in the form of matrix systems and in the form of membrane or pouch reservoir systems. Concerning the use of polymers, resins and other additives, there are no restrictions apart from the fact that the formulation must be compatible with the active ingredient zaleplon and that it must be suitable for delivering this active ingredient to the skin. In the simplest case, a TTS of the invention can be obtained by making a coarse (i.e. particulate), colloidal or molecular dispersion or solution of zaleplon in a solution of basic polymers, and coating this mixture onto a suitable substrate—usually a siliconized thermoplastic sheet (later the protective layer). After drying or evaporating off the solvent contents, the resulting layer, which represents the contact adhesive active ingredient reservoir, is covered with another sheet which later represents the later backing layer of the TTS. TTS can then be obtained from this laminate by punching out sheet-like structures in the required geometric shape.

Besides an active ingredient matrix, which may also be designed as reservoir in pouch form, the structure of the TTS of the invention comprises an active ingredient-impermeable backing layer and a likewise active ingredient-impermeable, detachable protective sheet.

Particularly suitable as backing layer are polyesters which are distinguished by particular strength, but also in addition virtually any other plastics which are compatible with skin, such as, for example, polyvinyl chloride, ethylene/vinyl acetate, vinyl acetate, polyethylene, polypropylene, cellulose derivatives and many others. In individual cases, the backing layer can be provided with an additional top layer, e.g. by vapor deposition of metals or other diffusion-blocking additives such as silica, alumina or similar substances known to the skilled worker.

The materials which can be used for the detachable protective sheet are the same as for the backing layer, provided that it is detachable through suitable surface treatment such as, for example, siliconization. However, it is also possible to use other detachable protective layers such as polytetrafluoroethylene-treated paper, cellophane, polyvinyl chloride, or the like.

Suitable basic materials for the preparation of the invention in the form of a TTS, in particular for producing the matrix layer(s), are, in particular, polymers based on acrylic acid and esters thereof, polyacrylates, isobutylene, ethylene/vinyl acetate, rubbers, styrene/diene copolymers, synthetic rubbers or hot melt adhesives. This list is far from complete but does reveal the broad applicability of the principle of the invention.

The TTS of the invention may additionally be such that the active ingredient is preferably present in the dissolved state, in which case,the formulation should, where possible, contain a solubilizer. It is possible to use for this purpose in particular polyhydric alcohols, particularly preferably 1,2-propanediol. Further examples of suitable solubilizers are tetrahydrofurfuryl alcohol, Transcutol, butanediol, glycerol, PEG 400, diethyltoluamide, monoisopropylideneglycerol.

The content of solubilizer based on the weight of the finished TTS can be between 1% and 50%, and preferably a content of between 5% and 35% by weight is chosen.

In order to achieve a high flux of active ingredient, it has also proved particularly advantageous to add to the active ingredient reservoir (and, where appropriate, also other layers of the TTS) a skin penetration enhancer. The concentration thereof is preferably in the range from 0.1% to 25%, particularly preferably in the range from 1% to 10%, in each case based on the weight of the TTS. Examples of suitable enhancers are decanol, dodecanol, oleic acid, oleic acid diethanolamide, myristic acid, sorbitan monolaurate, polyoxyethylene lauryl ethers, The polyoxyethylene lauryl ether Brij® is preferably employed. It is additionally possible for the zaleplon-containing TTS of the invention also to contain two or more enhancer compounds in combination.

In order to achieve a high rate of release, the aim is preferably to maximize the concentration of active ingredient in the active ingredient-containing layers. However, it must be noted in this connection that the physical stability may be impaired if the concentrations are too high.

The active ingredient concentrations used in the TTS of the invention are therefore in the range from 0.1% to 50%, preferably in the range from 1% to 10%, in each case based on the total weight of the active ingredient-containing layers.

In a particular embodiment, the TTS of the invention may also have two or more active ingredient-containing matrix layers. The individual matrix layers may moreover have different concentrations of active ingredient, enhancer or solubilizer. A further possibility is for the individual matrix layers also to have different contact adhesives. Unless the control of active ingredient release is brought about by other mechanisms, the reservoir can also be provided with a control membrane which controls the delivery of the active ingredient to the skin.

In another preferred embodiment, the active ingredient may also be present in a reservoir which is in pouch form and which is filled with a liquid, highly viscous, semisolid or thixotropic matrix. It is moreover particularly advantageous for the semisolid or thixotropic active ingredient reservoir to contain a gel former. The reverse side of the pouch facing away from the skin must in this case be impermeable to active ingredient, and the side facing the skin must be permeable to active ingredient. It is possible optionally for an active ingredient-permeable membrane (control membrane) to undertake the control of active ingredient release.

The method for producing the TTS of the invention can be, for example, as follows:

Firstly, the active ingredient zaleplon and a suitable enhancer, e.g. Brij® 30, are dissolved in a solubilizer, e.g. 1,2-propanediol, where the concentration of zaleplon should if possible reach the saturation solubility. The solution may, where appropriate, also be supersaturated. This solution is added to and dispersed by means of a suitable stirring apparatus in the silicone adhesive, which is likewise dissolved in a solvent, to result in a liquid/liquid dispersion which is as homogeneous as possible. This dispersion is coated uniformly with a suitable device onto a support sheet. Subsequently, by controlled drying, the solvent of the silicone adhesive and any solubilizer contents are removed. The laminate obtained in this way is then laminated with another sheet. Finally, TTS with a defined area are punched out and packed in an appropriate packaging.

The main area of use of the zaleplon-containing TTS of the invention is the treatment of sleep disorders, i.e. of disorders of initiating and maintaining sleep.

It is also possible according to the invention for zaleplon-containing TTS to be employed for other indications. For example, the zaleplon-containing TTS of the invention are suitable for the treatment of acute and chronic states of tension, agitation or anxiety, and for the treatment of conditions with increased muscle tone, e.g. for the treatment of prophylaxis of muscle spasms or muscle tenseness. The zaleplon-containing TTS can also be employed as premedication before surgical or diagnostic interventions, for assisting anesthesia, and as postoperative medication.

The zaleplon-containing TTS can also be employed for the prophylaxis or treatment of certain psychological disorders, e.g. for psychoses of the schizophrenic type, for anxiety states or for depressions. The invention likewise provides for the use of zaleplon-containing TTS for the treatment or prophylaxis of migraine or of epileptic seizures.

1. A zaleplon-containing pharmaceutical preparation, which preparation is a transdermal therapeutic system in plaster form, having a backing layer, having a contact adhesive active ingredient reservoir connected thereto, and having a protective layer which can be detached before application, where the active ingredient reservoir comprises zaleplon.

2. The preparation as claimed in claim 1, which comprises at least one solubilizer, preferably from the group of polyhydric alcohols, particularly preferably 1,2-propanediol.

3. The preparation as claimed in claim 1 or 2, which comprises at least one skin penetration enhancer, preferably from the group comprising polyoxyethylene fatty alcohol ethers, particularly preferably Brij® 30, and polyoxyethylene fatty acid esters, polyoxyethylene sorbitan fatty acid esters, sorbitan fatty acid esters, fatty acids, fatty alcohols, esters of fatty acids with methanol or ethanol or isopropanol, esters of fatty alcohols with acetic acid or lactic acid.

4. The preparation as claimed in claim 3, which comprises a combination of at least two penetration enhancers.

5. The preparation as claimed in any of claims 1 to 4, wherein the contact adhesive used for the contact adhesive active ingredient reservoir is selected from the group comprising silicone contact adhesives, contact adhesives based on polyacrylates, polyisobutylenes, polyterpenes, ethylene/vinyl acetate copolymers, rubbers, synthetic rubbers or hot melt adhesives, with use of combinations of said hot melt contact adhesives being preferred.

6. The preparation as claimed in one or more of the preceding claims, wherein the transdermal therapeutic system has a layered structure with at least two polymer matrix layers, with at least one of the matrix layers preferably comprising polymer constituents selected from the materials mentioned in claim 5.

7. The preparation as claimed in one or more of the preceding claims, wherein at least one matrix layer of the transdermal therapeutic system comprises polymer constituents selected from the group of substituted celluloses, preferably of methyl- or ethylcelluloses.

8. The preparation as claimed in one or more of the preceding claims, which comprises plasticizers in a concentration of from 0 to 30% by weight, preferably of 5-20% by weight, where the plasticizers are selected from the group comprising hydrocarbons, alcohols, carboxylic acids and derivatives thereof, ethers, esters and amines.

9. The preparation as claimed in one or more of the preceding claims, wherein the active ingredient reservoir is designed as reservoir which is in pouch form and which is filled with a liquid, highly viscous, semisolid or thixotropic matrix which comprises the active ingredient zaleplon.

10. The use of a zaleplon-containing transdermal therapeutic system in plaster form as claimed in one or more of claims 1 to 9 for the treatment of disorders of initiating or maintaining sleep.

11. The use of a zaleplon-containing transdermal therapeutic system in plaster form as claimed in one or more of claims 1 to 9 for the treatment of acute and chronic states of tension, agitation or anxiety.

12. The use of a zaleplon-containing transdermal therapeutic system in plaster form as claimed in one or more of claims 1 to 9 for the treatment of conditions with increased muscle tone.

13. The use of a zaleplon-containing transdermal therapeutic system as claimed in claim 12, is for the purpose of the therapy or prophylaxis of muscle spasms or muscle tenseness.

14. The use as claimed in any of claims 10 to 13, is for the purpose of premedication before surgical or diagnostic interventions, or for postoperative medication.

15. The use as claimed in any of claims 10 to 13, which is for the purpose of assisting anesthesia.

16. The use of a zaleplon-containing transdermal therapeutic system in plaster form as claimed in one or more of claims 1 to 9 for the treatment or prophylaxis of psychoses of the schizophrenic type.

17. The use of a zaleplon-containing transdermal therapeutic system in plaster form as claimed in one or more of claims 1 to 9 for the treatment or prophylaxis of depressions.

18. The use of a zaleplon-containing transdermal therapeutic system in plaster form as claimed in one or more of claims 1 to 9 for the treatment or prophylaxis of epileptic seizures.

19. The use of a zaleplon-containing transdermal therapeutic system in plaster form as claimed in one or more of claims 1 to 9 for the treatment or prophylaxis of migraine.

20. The use of zaleplon for producing a transdermal therapeutic system for the treatment of disorders of initiating or maintaining sleep by transdermal administration of the active ingredient zaleplon.

21. The use of zaleplon for producing a transdermal therapeutic system for the indications mentioned in claims 11-19.

22. A method for the treatment of disorders of initiating or maintaining sleep, which comprises the transdermal administration of the active ingredient zaleplon by means of a transdermal therapeutic system.

23. A process for producing a zaleplon-containing transdermal therapeutic system as claimed in any of claims 1 to 9, which comprises dissolving the active ingredient zaleplon and a skin penetration enhancer in a solubilizer, where the concentration of zaleplon should if possible reach the saturation solubility, and subsequently dispersing this solution by stirring in a contact adhesive solution, coating the resulting dispersion onto a support sheet and, after drying has taken place, laminating on another sheet, and finally punching out transdermal therapeutic systems with a defined area and packing them in packaging.
